(19) United States
(12) Patent Application Publication
(10) Pub. No.: US 2017/0234856 A1
(43) Pub. Date: Aug. 17, 2017

(54) DETECTION METHOD USING RECOMBINANT LIVING CELLS FOR DETECTING XENOBIOTIC SUBSTANCES AND ARRANGEMENT AND TEST KIT FOR PERFORMING THE DETECTION METHOD

(71) Applicant: Alfred-Wegener-Institut Helmholtz-Zentrum fuer Polar-und Meeresforschung, Bremerhaven (DE)

(72) Inventor: Ulf-Georg Bickmeyer, Bremerhaven (DE)

(21) Appl. No.: 15/502,787

(22) PCT Filed: Jul. 10, 2015
(86) PCT No.: PCT/DE2015/000352

§ 371 Date: Feb. 9, 2017

(30) Foreign Application Priority Data
Aug. 13, 2014 (DE) 10 2014 012 130.5

(51) Int. Cl. G01N 33/50; G01N 35/00; G01N 21/64; C12Q 1/68

(52) U.S. Cl. CPC G01N 33/5014; C12Q 1/6897; G01N 35/00871; G01N 21/6428; G01N 2333/43504; G01N 2021/6439

(57) ABSTRACT

Dynamic expression behavior of recombinant living cells is used and detects translocation in an integral manner, preferably using a cell-inherent defense system, preferably gene sequences, aᵢ-mᵢ, in recombinant cells in a test kit for performing the detection method code for transporter proteins, Aᵢ, with fluorescent marker proteins, Mᵢ, as fusion constructs, Aᵢ-Mᵢ. The cell-inherent defense system is activated under the influence of foreign substance. Transporter proteins, Aᵢ, that transport the foreign substance out of the living cells are expressed in an increased manner. Together with the transporter proteins, Aᵢ, the marker proteins, Mᵢ, are also expressed in an increased manner, which is optically detected and enables corresponding conclusions about the foreign substance. The detection method is preferably implemented using an arrangement of genetically modified aquatic organisms or living cells thereof, which are permanently exposed in a water-permeable habitat tank in an aquatic system in the vicinity of a technical installation.

### CROSS-REFERENCE TO RELATED APPLICATIONS

This application is a U.S. national stage application under 35 U.S.C. §371 of International Application No. PCT/DE2015/000352, filed on Jul. 10, 2015, and claims benefit to German Patent Application No. DE 10 2014 012 130.5, filed on Aug. 13, 2014. The International Application was published in German on Feb. 18, 2016, as WO 2016/023530 A1 under PCT Article 21(2).

### FIELD

The invention relates to a detection method using recombinant living cells for detecting xenobiotic substances.

### BACKGROUND

Xenobiotic substances (foreign substances) are chemical compounds which are foreign to the biological metabolic cycle of an organism. They contain in part structural elements which do not occur or occur only extremely rarely in that form in natural substances. In nature, xenobiotics are frequently of anthropogenic origin, for example synthetic plant protection agents, halogenated hydrocarbons or plastics materials. Many of these materials are biodegradable only with difficulty or not at all. Xenobiotics can have a positive effect, no effect at all or a harmful effect on the environment and organisms. A harmful toxic effect depends, as well as on ingestion, also on biodegradability and on accumulation in specific organisms or parts of organisms.

Xenobiotic substances can occur in solid, liquid or gaseous form in systems, as well as in all three states of aggregation. In aquatic systems in particular, for example rivers, lakes or oceans, there often occur anthropogenic and biogenic xenobiotic substances, the precise chemical nature of which is unknown and/or the concentration of which is so low that they are not toxicologically relevant. Nevertheless, knowledge thereof is often helpful, if not even absolutely essential. The known chemical analyses with their toxicological tests using model organisms and cells and, in the broadest sense, the known eco-tests generally function only at higher concentrations and also provide only few indications of the substance class of the chemicals. This accordingly gives rise to the particular problem, in particular in the case of aquatic systems, of how such substances can be detected quickly and reliably.

DE 698 24 763 T2 describes a detection method which permits the identification of a toxic substance which directly or indirectly influences the translocation of a biologically active polypeptide. In this case, the biologically active polypeptide is a polypeptide that influences intracellular processes following activation of an intracellular signal transduction pathway. The intracellular signal transduction pathway includes an enzymatic reaction and is a coordinated intracellular process in which a living cell converts an external or internal signal into cellular responses. One or more cells that exhibit stable expression of a nucleic acid coding for a hybrid polypeptide are cultivated. In this case, the hybrid polypeptide comprises a fluorophore, in particular a green fluorescent protein GFP, which is coded for by a nucleic acid that is linked to the biologically active polypeptide or part thereof. The cells are incubated with a toxic substance that is to be screened for the biological function or biological activity. The light emitted by the fluorophore in the incubated cells is detected in respect of changes. Such changes are changes of location, that is to say changes of location of the hybrid polypeptide in the cell are detected in a spatially resolved manner by means of the fluorescent markings. Such detected changes of location indicate a change in the translocation of the biologically active polypeptide in the incubated cells and thus the interaction thereof with specific toxic substances. The known detection method tests cell systems in which an intracellular translocation of a biologically active polypeptide takes place through the influence of a toxic substance and provides a rapid and reproducible quantification of the translocation responses. As a result, it is possible to draw meaningful conclusions about the influences on cellular systems and the translocation responses thereof. However, stable expression of the hybrid is absolutely essential for this purpose, in order to be able to detect such a translocation using the known detection method. In summary, the detection method according to DE 698 24 763 T2 is accordingly applicable only to translocating substances which initiate an intracellular response and performs a spatially resolved detection of marker proteins which is based on stable expression of the fusion proteins used.

A similar method is known from DE 696 35 355 T2. Here too, hybrid polypeptides marked with GFP are produced in order to characterize the biological activity of a sample. However, there is no localization of the fluorescence, but its intensity is measured and compared with normal values.

WO 95/07463 A1 also describes a cell which is capable of expressing GFP, and a method of detecting a protein of interest in a cell, which method is based on introducing into a cell a gene that has a gene sequence section that links the protein of interest to a gene sequence section coding for a GFP, so that the protein produced by the gene contains the protein of interest fused to the GFP. After cultivation of the cells, the fluorescence is then localized in the cell, whereby the protein of interest is also localized in the cell. Accordingly, here too, intracellular processes are again localized by fluorescence detection. WO 95/07463 A1 further describes cells which are used for detecting molecules, such as hormones or heavy metals, in a biological probe. Living organisms may exhibit those cells. The regulatory element of the gene, which is influenced by the molecule of interest, is operatively linked to a GFP. The regulatory element can be a promoter belonging to a gene that is responsible for cell viability. The presence of the molecules influences the regulatory element, which in turn influences the expression of the GFP. In this manner, the gene coding for GFP is used as a reporter gene in a cell which is designed to monitor the presence of a specific molecular identity. Accordingly, with this known method, the effects of stress, for example, on the living organism can be indicated by increased fluorescence.

It is known implicitly from DE 698 24 763 T2 mentioned above that functional proteins fused with a marker protein are not impaired in terms of their function in the fusion protein and can be expressed unchanged. The same is also known from the publication “Structural requirements for the apical sorting of multidrug resistance protein 2 (ABCC2)” by A. T. Nies et al. (in Eur. J. Bio chem. 269, 1866-1876 (2002)). In this publication it is shown that GFP couplings to MRP transporter proteins in principle function correctly and the transporter functions are not disrupted. The transporter proteins also work properly with the marking. However, it is also an aim of this publication to detect the localization of the transporters and to determine the portion of the transporters that is responsible for localization to specific sites. For the coupling of GFPs to human MDR proteins, the same is known from the publication “Sensitive and Specific Fluorescent Probes for Functional Analysis of the Three Major Types of Mammalian ABC Transporters” by I. V. Lebedeva et al. (in PLoS ONE July 2011, Vol. 6, Issue 7, e22429, pp. 1 to 12). Here, the GFPs are used in a flow cytometry measurement as indicators of MDRs in living cells.

ABC transporters form a large family of membrane proteins which possess an ATP (adenosine triphosphate)-binding cassette (ABC acronym of ATP binding cassette) as a common structural element and actively transport chemical substrates across a cell membrane. Even when they occur in toxicologically irrelevant concentrations, chemical substances can cause a reaction in living organisms and their cells by activating or upregulating the first cellular defense system, to which the ABC transporters belong. Living organisms can accordingly activate a protection mechanism which, as required, is able to remove foreign substances from the cell interior and then also from entire tissues, with the consumption of energy. Multiple drug resistance proteins (MDR) and multidrug resistance-related proteins (MRP) above all are involved in the transport. These names describe the phenomenon that cells have or develop resistance to specific substances.

The publication “Reporter Dyes Demonstrate Functional Expression of Multidrug Resistance Proteins in the Marine Flatworm Macrostomum lignano: The Sponge-Derived Dye Ageladine A Is Not a Substrate of These Transporters” by K. Tietje et al. (in March Drugs 2013, 11, 3951-3969) reports in detail on MDR and MRP transporter proteins in the flatworm Macrostomum lignano in connection with incubation with the fluorescent dye ageladine A.

Green fluorescent protein (GFP) is a protein from the jellyfish Aequorea victoria which was first described in 1961 by Osamu Shimomura and fluoresces green when excited with blue or ultraviolet light. Modified variants of GFP exhibit different color spectra. Its invaluable importance in biology, in particular cell biology, lies in the possibility of fusing GFP gene-specifically with any other proteins. By means of the fluorescence of the GFP, the spatial and temporal distribution of the other protein in living cells, tissues or organisms can thus be observed directly. The GFPs and the nature thereof are described in detail in the publication “Nobel lecture: constructing and exploiting the fluorescent protein paintbox” by R. T. Tsien (in Integr. Biol. 2010, 2, 77-93). Newer variants of the GFP are described, for example, in DE 696 04 298 T2.

### SUMMARY

An aspect of the invention provides a method for detecting xenobiotic substances, the method comprising: contacting one or more recombinant living cells with one or more xenobiotic substances so as to effect, via at least one promoter, P1, which is active in the living cells, an activation or upregulation of an expression of at least one gene sequence section, ai, which codes for at least one functional protein, Ai, of known function. The gene sequence section, ai, is recombinantly modified by fusion with at least one marker protein gene, mi, the marker protein gene coding for at least one marker protein, Mi, with one or more known marker properties. The marker protein gene does not influence coding of the functional protein, Ai, and a gene fusion construct, ai-mi, codes for a fusion protein, Ai-Mi, of the at least one functional protein, Ai, and the at least one marker protein, Mi. An increased occurrence of the at least one marker protein, Mi, as a cell reaction is determined integrally by detection of its marker properties.

### BRIEF DESCRIPTION OF THE DRAWINGS

The present invention will be described in even greater detail below based on the exemplary figure. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawing which illustrate the following:

FIG. 1 a schematic of an embodiment of the invention.

### DETAILED DESCRIPTION

An aspect of the invention provides a method using recombinant cells having at least one gene sequence section aᵢ, wherein the gene sequence section aᵢ is recombinantly modified by fusion with at least one marker protein gene mᵢ. The effects of the xenobiotic substance on the expressed gene fusion product aᵢ-mᵢ are detected via the marker protein gene mᵢ that is present. The invention relates further to an arrangement and to a test kit that comprises a means, based on recombinant living cells, for performing the detection method.

The present invention provides a method by means of which the presence of as many xenobiotic substances as possible can be detected reliably, simply and quickly. This is also intended to be possible when the precise chemical nature of the substances is not known or when the substances occur in concentrations such that they are not toxicologically relevant. An arrangement for performing the detection method is to be further provided, by means of which the method can be put into actual practice particularly simply and reliably and, moreover, independently. A test kit is thereby to be used which ideally complements the arrangement and makes the applicability of the detection method particularly varied by providing different recombinant cells. These objects are achieved by the method claim and the two product claims. Advantageous modifications of the ways of achieving the objects are described in the respective dependent claims and are explained in greater detail hereinbelow in connection with the invention.

There is claimed according to the invention a detection method using recombinant living cells for detecting those xenobiotic substances which, when they come into contact with the living cells, effect, via at least one promoter which is active in the living cells, an activation or upregulation of the expression of at least one gene sequence section aᵢ which codes for at least one functional protein Aᵢ of known function, wherein the gene sequence section aᵢ is recombinantly modified by fusion with at least one marker protein gene mᵢ which codes for at least one marker protein Mᵢ with known marker properties which does not influence the coding of the functional protein Aᵢ, and the gene fusion construct aᵢ-mᵢ codes for a fusion protein Aᵢ-Mᵢ of the at least one functional protein Aᵢ and the at least one marker protein Mᵢ, and wherein an increased occurrence of the at least one marker protein Mᵢ as a cell reaction is determined integrally over the surface by detection of its marker properties. i thereby denotes the continuous index with i=from 1 to n for different gene sequence sections aᵢ, functional proteins Aᵢ, marker protein genes mᵢ and marker proteins Mᵢ.

The method according to the invention can be used as a very efficient procedure for testing or detecting the influence of a xenobiotic substance on a physiological process, in particular in conjunction with the testing of substances for toxicity. It differs from the known detection method especially in that a dynamically changeable expression of the fusion protein Aᵢ-Mᵢ forms the basis of the measurement. An increased formation of the marker protein Mᵢ is detected integrally over a surface and reliably indicates that the functional protein Aᵢ is also being formed in an increased manner. Foreign substances in the living cells much more frequently cause a more pronounced expression of individual functional proteins than a translocation of proteins, which is a very much more complex cell operation. The focus of the invention is accordingly on dynamic expression as sensor signal, while expression in the known detection method is assumed to be constant and constitutes a fundamental requirement. The basic active principle underlying the invention is that of influencing the expression of different proteins in living cells in the sense of change on the basis of xenobiotic substances. In the invention, dynamic expression is understood as being a foreign substance signal, which is the cellular response to contamination. In the invention, the living transgenic cells are used as sensors, the cellular processes and the conversion thereof into a cell response being used in the detection method in the sense of message processing and transmission. The invention provides a detection method using sensor technology based on living cells, which performs the decisive step from the theoretical research laboratory into practical aquatic reality.

The detection method according to the invention provides an analysis of the cell responses which is integral over the surface and not—as in the prior art—spatially resolved, it being possible for the integral detection to be performed quickly, simply and reliably. Long incubation times of the transgenic cells are not necessary, and therefore the detection method according to the invention is also highly suitable for rapid flow measurements. An integral detection is also very much more simple to perform than a spatially resolved detection, which also always requires the transgenic cells to be individually observable. Time-consuming singularization procedures are necessary therefor. In the case of the integral detection in the detection method according to the invention, the cell response of the cells can simply be observed in the cell structure and evaluated quantitatively. It is accordingly also possible to automate the detection method in an independent measuring system without difficulty.

In the signal pathways between the foreign substance and the transgenic cell that are used for the detection method according to the invention, the substance to be detected acts on corresponding promoters in the recombinant living cell and activates or upregulates the gene expression thereof, which describes the characteristic or activity state of one or more genes. In genetics, a promoter is a nucleotide sequence on the DNA that permits the regulated expression of a gene. The promoter is an essential part of a gene. It is located at the 5′ end of the gene (head end) and thus upstream of the RNA-coding region. The most important property of a promoter is the specific interaction with specific DNA-binding proteins which mediate the start of transcription of the gene by the RNA polymerase (transcription factors).

Using the detection method according to the invention it is possible to detect in principle all xenobiotic substances which, when they come into contact with non-recombinant living cells, effect, via at least one promoter which is active in the living cells, an activation or upregulation of the expression of at least one gene sequence section. The use of the detection method according to the invention can yield results which are purely of an informal nature, for example it may be of interest to determine the sites at which specific, in particular anthropogenic, foreign substances accumulate in different systems. It may also be of interest to monitor whether xenobiotic substances occur at all in a natural or technical system, even if those substances are initially of unknown nature. Another use of the detection method according to the invention consists in warning a person of the occurrence of substances which are harmful or even toxic to him. Accordingly, it is preferably and advantageously provided in the detection method according to the invention that xenobiotic substances which are of toxic and/or unknown nature and/or which occur in concentrations that are not toxicologically relevant are detectable. Particularly advantageously and preferably, xenobiotic substances in the vicinity of technical installations are detectable by the detection method according to the invention. The detection method according to the invention is accordingly particularly suitable for the operation of warning systems in the vicinity of such technical installations. Operation in the vicinity of refineries, chemical plants and power stations is particularly expedient in order to be able to detect toxic substances, such as heavy metals or halogenated hydrocarbons, quickly and reliably. Depending on the nature of the xenobiotic substances, different proteins are activated in the recombinant living cells and are detectable by their genetic marking.

A requirement for the applicability of the detection method according to the invention is the addressing of at least one promoter in the living cells by the xenobiotic substance that is to be detected, the addressed promoter then activating or upregulating a specific gene sequence section as the reaction. Contact with the xenobiotic substance activates a cell reaction. It is conceivable that different xenobiotic substances occur at one and the same time and address different promoters in the cells as a reaction, so that different gene sequence sections are then activated or upregulated. It is therefore advantageous and preferred if different gene sequence sections aᵢ which code for different functional proteins Aᵢ are recombinantly modified with marker protein genes m, which code for marker proteins Mᵢ with different marker properties, and the gene fusion constructs aᵢ-mᵢ code for different fusion proteins Aᵢ-Mᵢ a conclusion being drawn about the increased coding for the corresponding functional proteins Aᵢ by detection of the different marker properties. It is then possible to draw conclusions about the type and nature of the different xenobiotic substances via the functional proteins Aᵢ which are correspondingly produced in an increased manner. The contaminating xenobiotic substances can thereby initiate different cell reactions depending on their chemical properties.

A particularly important and frequently occurring cell reaction is activation of the cell-inherent defense system. Accordingly, it is particularly advantageous and preferred if the at least one gene sequence section aᵢ that is activated or upregulated in terms of its expression by xenobiotic substances is a gene sequence section which codes for the defense and detoxification mechanisms of the living cells, the encoded functional protein Aᵢ in the fusion proteins Aᵢ-Mᵢ being a member of the family of the ABC transporter proteins, the function of which is the membrane transport of foreign substances. The xenobiotic substances to be detected accordingly stimulate the first cellular defense system of the recombinant cells and lead to the detectable cell reactions. Since this cell-inherent defense system is very broad, a very large number of xenobiotic substances can be detected by the detection method according to the invention. In particular, a very large number of harmful toxic substances can be detected safely and reliably—even in extremely low concentrations—because the cell-inherent defense system—without great differences between the different donor organisms—is consequently designed for defense against precisely such substances.

In particular, it is preferred and advantageous if the gene sequence section aᵢ codes for a functional protein Aᵢ in the fusion proteins Aᵢ-Mᵢ in the form of a multidrug resistance protein (MDR transporter protein) and/or of a multidrug resistance-associated protein (MRP transporter protein) from the family of the ABC transporter proteins. These are the transporter proteins from the large family of the ABC transporters that occur most frequently and are involved most intensively in the cell-inherent defense system. It has already been stated above that, in the detection method according to the invention, different gene sequence sections aᵢ which code for different functional proteins Aᵢ can be fused with different marker protein genes mᵢ which code for different marker proteins Mᵢ with different marker properties (see also below). Accordingly, increased expression of MDR transporter proteins and MRP transporter proteins can reliably be detected by the detection method according to the invention. It is therefore possible that, in the detection method according to the invention, it is concluded in the case of an increased occurrence of MDR transporter proteins as the cell reaction that a xenobiotic substance having uncharged molecules with chain lengths in the same order of magnitude is present and in the case of an increased occurrence of MRP transporter proteins as the cell reaction that a xenobiotic substance having charged molecules with chain lengths between 100 Da (unit daltons) and 8 kDa, preferably between 1 and 3 kDa, particularly preferably between 1.5 and 2.5 kDa, for example up to 2 kDa, is present. By means of such a classification, the xenobiotic substance, in particular when it is an unknown toxic substance, can already be limited in terms of its origin.

Marker proteins can have a very wide variety of different marker properties of chemical and physical nature, which are correspondingly detectable. For example, marker proteins can measurably change the pH of their surroundings. However, optical detection of the marker proteins, for example detection of the size, is simpler. The simplest detection, however, is color recognition, in particular if the marker protein fluoresces on its own or when excited. Accordingly, it is advantageous and preferred in the detection method according to the invention if marker proteins Mᵢ having fluorescence as the marker property are used, at least the fluorescence wavelength and/or the fluorescence intensity being detected. Marker proteins Mᵢ from the family of the green fluorescent proteins (GFP) or their fluorescent homologs or derivatives or mutants of those GFPs can preferably and advantageously be used. The GFPs have been well researched and are easy to handle and are available in many color variants.

The modified detection method according to the invention can accordingly include the color marking, on the basis of the GFPs and the color modifications thereof, of different ABC transporters in living transgenic recombinant cells in order, depending on the dynamic degree of expression thereof, to effect a detection of the existence of anthropogenic and biogenic foreign substances in water. The fluorescent protein GFP originating from jellyfish of the type Aequoria, and modifications thereof, is inserted into the genome of the living cells, and the proteins are expressed together with the ABC transporters. If the ABC transporters are expressed in an increased manner, the GFP is also expressed in an increased manner and the fluorescence is accordingly increased. Recombinant cells or organisms (see below) which react to environmental changes with increased expression of MDR/MRP and thereby increase the fluorescence in the chosen GFP region are produced.

The detection method according to the invention is based on the reactions of recombinant living cells to contamination with xenobiotic substances. In principle, the cells can be contaminated with a xenobiotic substance in all three states of aggregation. In addition to contamination of the living cells with a solid or gaseous foreign substance, the foreign substance to be detected can in particular also be distributed or dissolved in an aqueous solution, so that contamination of the living cells in aqueous phase occurs. In addition to contamination in a body of water, applications in the medical field, for example in connection with blood, are also conceivable here. In the case of aqueous contamination, living cells, or entire cell cultures, can in principle be kept in corresponding nutrient solutions. It is thereby advantageous and preferred if the living cells originate from an aquatic organism which already has the living cells or from a non-recombinant aquatic mother organism, which organism can preferably be a crustacean, a cnidarian, a sea anemone, a spiny creature, a sponge or a round- or flat-worm, particularly preferably the flatworm Macrostonum lignano. In the second case, living cells are taken from the mother organism and genetically modified within the meaning of the invention in the laboratory.

If a living aquatic organism which already contains the recombinant cells is used, it is advantageous and preferred if at least a complete living aquatic organism which has the living cells is used. The genetically modified organism can again preferably be a crustacean, a cnidarian, a sea anemone, a spiny creature, a sponge or a round- or flat-worm, particularly preferably the flatworm Macrostonum lignano. Such simple aquatic organisms are relatively simple to keep. It is advantageous if the aquatic organisms used are already genetically decoded at least in part (which is the case with the flatworm Macrostonum lignano, many EST—expressed sequence tags—are already known here), so that the recombinant modification of the specific gene sequence sections aᵢ with the marker protein genes mᵢ can routinely be performed by specialists. The living cells with the fusion construct aᵢ-mᵢ can colonize and multiply anywhere in the contaminated organism, in particular also on the surface or the skin of the organism. Marking fluorescence phenomena under contamination can then easily be detected there in respect of color and intensity. However, in order to be able reliably to detect the fluorescent markers in the whole organism, it is particularly advantageous and preferred if at least one organism that is at least partially transparent is used. The aquatic organism in the form of the flatworm Macrostonum lignano is largely transparent, so that fluorescence phenomena in its interior are also readily detectable. In the case of contamination of the recombinant cells with solid or gaseous foreign substances, whole organisms can likewise be used, which then exhibit correspondingly detectable reactions, for example, on the skin or other outer regions or in the lungs or other internal organs.

In the case of the detection of emissions, a detection in respect of their color and intensity that is performed in an automated manner as an optical detection is naturally preferred and advantageous. In the case of other marker properties that are to be detected, other detection principles are used. For example, pH changes can be detected particularly well with the fluorescent dye ageladine A, in particular also in transparent organisms. Detections based on electrical parameters, such as current and voltage, can likewise be used if the marker properties have an influence on current and voltage. Optical detection is, however, contactless and can also be performed particularly well in an automated manner as a simple observation.

Suitable arrangements for performing the detection method according to the invention are governed by the type of contamination, in particular by the state of aggregation thereof. In the case of solid and gaseous contamination, an entire organism can be kept in air; cell cultures are to be kept in aqueous solutions. A claimed arrangement in the case of a liquid, in particular aqueous, contamination is described hereinbelow. The claimed arrangement according to the invention for performing the detection method according to the invention using living cells of aquatic organisms is characterized by a habitat tank for keeping in the live state at least one cell culture or at least one complete living aquatic organism in an aquatic system, having a water-permeable wall which permits water exchange with the water of the surrounding aquatic system, and a closable opening for introducing and removing the living cell culture or the living aquatic organism, by a feed system for automatically feeding the living cell culture or the living aquatic organism, and by an automatic detection system having a detector for monitoring the marker properties of the fused marker proteins in the cells of the living cell culture or of the living aquatic organism, as well as a data station at least for storing the detected data and a transmitting station for transmitting the detected data to an external station. In the case of optical detection, an optical detection system, in particular for fluorescence detection in the case of fluorescent marker properties, is correspondingly preferably and advantageously used. However, the genetically modified cells or complete aquatic organisms used are not released at any time but live in the closed tanks, which permit water exchange at any time.

In addition, an optical signaling device can preferably and advantageously be provided, which signaling device can be activated automatically depending on the detected data and, when activated, is visible at the location of the habitat tank, and/or a monitoring device on the habitat tank for monitoring the live state of the aquatic organisms is used. Such systems can then be used particularly successfully as independent early warning systems in the vicinity of technical installations where an emission of xenobiotic substances is to be expected, in order to be able to warn people who are in that location in a simple manner. Such systems themselves operate wholly harmlessly in biological terms and in an environmentally friendly manner.

In addition to the arrangement, there is also claimed a test kit for performing the method, having a means based on recombinant living cells for performing the detection method, which test kit is characterized by a configuration in the form of living cells having at least one gene fusion construct aᵢ-mᵢ of a gene sequence section aᵢ which codes for at least one functional protein Aᵢ from the family of the ABC transporter proteins, and a marker protein gene mᵢ which codes for at least one marker protein Mᵢ from the family of the green fluorescent proteins which does not influence the coding of the functional protein Aᵢ, the gene fusion construct aᵢ-mᵢ coding for a fusion protein Aᵢ-Mᵢ of the functional protein Aᵢ and the marker protein Mᵢ. Transporter proteins from the defense system of a living cell which are recombinantly modified and marked with a fluorescent GFP marker protein are not known from the prior art and have consequently not hitherto been used as sensors for detecting harmful substances. Preferably and advantageously, the living cells used are characterized in that they are derived recombinantly from a non-recombinant aquatic mother organism, preferably from a crustacean, a cnidarian, a sea anemone, a spiny creature, a sponge or a round- or flat-worm, particularly preferably from the flatworm Macrostonum lignano. Furthermore, the living cells can preferably and advantageously be recombinantly derived from non-recombinant cell cultures or non-recombinant immortalized cell lines. Such cell cultures and cell lines can be deposited in publicly accessible collections of microorganisms and cell cultures. The same is true for cell cultures and cell lines having the recombinant cells. Further details of the invention will be found in the following embodiment.

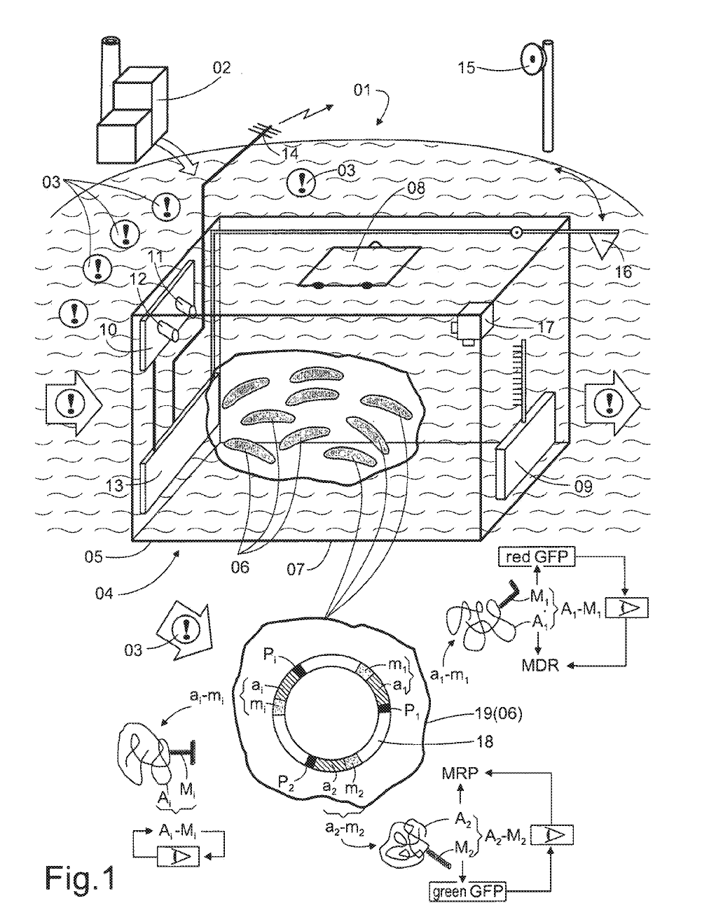

The detection method and the arrangement for performing the detection method according to the invention, and advantageous modifications thereof, will be described in greater detail, for better understanding of the invention, with reference to the schematic FIG. 1, which is not to scale, using an embodiment. In this case, the embodiment could be headed “Color coding of xenobiotic chemical structures by fluorescent marine organisms”.

FIG. 1 shows an aquatic system 01, which in the example shown is a bay. At the edge of the bay there is a technical installation 02, which in the example shown is a chemical plant. Xenobiotic substances 03 (symbol exclamation mark) are introduced into the aquatic system 01 from the technical installation 02. People bathe in the bay. An early warning of harmful substances is accordingly extremely expedient.

An arrangement 04 for performing the detection method according to the invention is shown in the foreground. There is shown a habitat tank 05 for keeping in the live state a plurality of aquatic organisms 06 (in the cutaway portion), which in the example shown are flatworms of the genus Macrostonum lignano, which are arranged in the aquatic system 01. These flatworms are largely transparent and allow fluorescence phenomenon on their interior to be detected without difficulty. The habitat tank 05 has a water-permeable wall 07. This permits unimpeded water exchange with the water of the surrounding aquatic system 01. In the wall 07 there is a closable opening 08 for introducing and removing the aquatic organisms 06 and a feed system 09 for automatically feeding the aquatic organisms 06. Furthermore, an automatic detection system 10 is located in the habitat tank 05, which detection system has a light source 11 for irradiating the aquatic organisms 06 and a detector 12, which in the embodiment shown is an optical detector, for monitoring the aquatic organisms 06 in the sense of detecting their fluorescence emission. There are further provided a data station 13, which has a battery-assisted or external power supply, at least for storing the detected data, and a transmitting station 14 for transmitting the detected data to an external receiving station 15. An optical signaling device 16 is further arranged on the habitat tank 05, which signaling device can be activated automatically depending on the detected data and, when activated, is visible at the location of the habitat tank 05 as a warning of contamination by xenobiotic, in particular toxic, substances. Finally, a monitoring device 17 is also provided on the habitat tank 05 for monitoring the live state of the aquatic organisms 06 used. This can be a video camera, for example.

The genetic modification of the living cells of the aquatic organisms 06, in order to allow them to be used in the detection method according to the invention, is shown in the bottom enlarged section in FIG. 1. There is shown, in a recombinant living cell 19, a plasmid 18 having a gene sequence section a₁ which codes for a functional protein A₁, which in the chosen embodiment is an MDR transporter protein, and having a gene sequence section a₂ which codes for a functional protein A₂, which in the chosen embodiment is an MRP transporter protein. Both transporter proteins come from the family of the ABC transporter proteins. The gene sequence section a₁ is activated or upregulated by a promoter P₁, and the gene sequence section a₂ is activated or upregulated by the promoter P₂. The gene sequence section a₁ is recombinantly modified with a marker gene sequence m1, and the gene sequence section a₂ is recombinantly modified with a marker gene sequence m₂. The gene sequence m₁ in the chosen embodiment codes for a green fluorescent marker protein M₁, and the marker gene sequence m₂ in the chosen embodiment codes for a red fluorescent marker protein m₂. Both marker proteins come from the family of the GFP. The gene fusion construct of the gene sequence section a₁ and the marker gene sequence m₁ is denoted a₁-m₁. It codes for the marked fusion protein A₁-M₁. The gene fusion construct of the gene sequence section a2 and the marker gene sequence m₂ is denoted a₂-m₂. It codes for the marked fusion protein A₂-M₂. The designations with the continuous index i indicate that further gene sequence sections aᵢ can be recombinantly modified by further markers mᵢ which then code for different functional proteins Aᵢ and different marker proteins Mᵢ with different marker properties in different fusion constructs Aᵢ-Mᵢ.

In the embodiment shown, when the plasmid 18 of the aquatic organism 06 comes into contact with the xenobiotic substance 03, the cell-inherent defense system is activated via the promoter P₁. This results in the increased production of MDR proteins A₁ and thus marker proteins M₁, which fluoresce green when activated. The green fluorescence emission which occurs or is increased is detected by means of the detection system 10. The data are transmitted to the external receiving station 15 and evaluated. At the same time, the optical signaling device 16 is activated and the people in and in the vicinity of the aquatic system 01 are warned reliably and in good time.

By means of the detection system according to the invention based on recombinant gene sequences in the genome of a genetically modified cell which, when it comes into contact with a xenobiotic substance, generates a cell response in the form of a dynamic gene expression, it is accordingly possible to construct a relatively simple and reliable warning system for people. In particular, it is also possible to detect contaminations which other detection methods fail to detect because the concentrations are too low. Together with the arrangement and the test kit for performing the detection method according to the invention, which is based in particular on keeping genetically modified, living, complete aquatic organisms in an aquatic system, it is possible to provide a preferred complex system for practice which reliably detects and warns against different xenobiotic substances in a very wide variety of situations.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B, and C” should be interpreted as one or more of a group of elements consisting of A, B, and C, and should not be interpreted as requiring at least one of each of the listed elements A, B, and C, regardless of whether A, B, and C are related as categories or otherwise. Moreover, the recitation of “A, B, and/or C” or “at least one of A, B, or C” should be interpreted as including any singular entity from the listed elements, e.g., A, any subset from the listed elements, e.g., A and B, or the entire list of elements A, B, and C.

### LIST OF REFERENCE SIGNS

01 aquatic system

02 technical installation

03 xenobiotic substance

04 arrangement for performing the detection method

05 habitat tank

06 complete living aquatic organism (genetically modified) which has living cells 19

07 water-permeable wall

08 closable opening

09 feed system

10 automatic detection system

11 light source

12 detector

13 data station

14 transmitting station

15 external receiving station

16 optical signaling device

17 monitoring device

18 plasmid

19 recombinant living cell

aᵢ gene sequence section, i=1..n

mᵢ marker gene sequence

aᵢ-mᵢ gene fusion construct

Aᵢ functional protein

Mᵢ marker protein

Aᵢ-Mᵢ fusion protein

Pᵢ promoter

1. A method for detecting xenobiotic substances, the method comprising:
contacting one or more recombinant living cells with one or more xenobiotic substances so as to effect, via at least one promoter, P₁, which is active in the living cells, an activation or upregulation of an expression of at least one gene sequence section, aᵢ, which codes for at least one functional protein, Aᵢ, of known function,
wherein the gene sequence section, aᵢ is recombinantly modified by fusion with at least one marker protein gene, mᵢ, the marker protein gene coding for at least one marker protein, Mᵢ, with one or more known marker properties,
wherein the marker protein gene does not influence coding of the functional protein, Aᵢ, and a gene fusion construct, aᵢmᵢcodes for a fusion protein, Aᵢ-Mᵢ, of the at least one functional protein, Aᵢ, and the at least one marker protein, and
wherein an increased occurrence of the at least one marker protein, Mᵢ, as a cell reaction is determined integrally by detection of its marker properties.

2. The method of claim 1, wherein the xenobiotic substances are of toxic and/or unknown nature and/or which occur in concentrations that are not toxicologically relevant are detectable.

3. The method of claim 1, wherein the xenobiotic substances in the vicinity of technical installations and/or in aquatic systems are detectable.

4. The method of claim 1, wherein different gene sequence sections, aᵢ, which code for different functional proteins, Aᵢ, are recombinantly modified with marker protein genes, mᵢ, which code for marker proteins, Mᵢ, with different marker properties, and the gene fusion constructs, aᵢ-mᵢ , code for different fusion proteins, Aᵢ-Mᵢ, a conclusion being drawn about the increased coding for the corresponding functional proteins Aᵢ by detection of the different marker properties.

5. The method of claim 1, wherein the at least one gene sequence section, aᵢ, that is activated or upregulated in terms of its expression by a xenobiotic substance is a gene sequence section which codes for defense and detoxification mechanisms of the living cells,
wherein the encoded functional protein, Aᵢ, in the fusion proteins, Aᵢ-Mᵢ, is a member of the family of the ABC transporter proteins, the function of which is membrane transport of foreign substances.

6. The method of claim 5, wherein the gene sequence section, aᵢ, codes for a functional protein Aᵢ, in the fusion proteins, Aᵢ-Mᵢ, in the form of a multidrug resistance protein (MDR transporter protein) and/or of a multidrug resistance-associated protein (MRP transporter protein) from the family of the ABC transporter proteins.

7. The method of claim 6, comprising concluding, in the case of an increased occurrence of MDR transporter proteins as a cell reaction, that a xenobiotic substance including uncharged molecules with chain lengths in the same order of magnitude is present, and, in the case of an increased occurrence of MRP transporter proteins as the cell reaction, that a xenobiotic substance including charged molecules with chain lengths between 100 Da and 8 kDa is present.

8. The method of claim 1, wherein the marker proteins, Mᵢ, have fluorescence, and
wherein at least the fluorescence wavelength and/or the fluorescence intensity are detected.

9. The method of claim 8, wherein the marker proteins, Mᵢ, are from the family of the green fluorescent proteins (GFP) or their fluorescent homologs or derivatives or mutants of those GFPs.

10. The method of claim 1, wherein the living cells originate from an aquatic organism which already has the living cells or from a non-recombinant aquatic mother organism.

11. The method of claim 1, wherein at least a complete living aquatic organism including the living cells is used.

12. The method of claim 11, wherein the aquatic orgasnism is at least partially transparent.

13. The method of claim 1, comprising detecting in an automated manner as an optical detection.

14. An arrangement for performing the method of claim 1, comprising:
a habitat tank, configured to keep in the live state at least one cell culture with the living cells or at least one complete living aquatic organism including the living cells in an aquatic system;
a water-permeable wall, configured to permit water exchange with water of a surrounding aquatic system;
a closable opening, configured to introduce and remove the cell culture or the aquatic organism using a feed system configured, to automatically feed the cell culture or the aquatic organism;
an automatic detection system including a detector configured to monitor marker properties of the fused marker proteins in the cells of the cell culture or of the aquatic organism;
a data station at least for storing detected data; and
a transmitting station, configured to transmit the detected data to an external station.

15. The arrangement of claim 14, comprising an optical signaling device which can be activated automatically depending on the detected data,
wherein, when activated, the optical signaling device is visible at the location of the habitat tank, and/or a monitoring device on the habitat tank for monitoring the live state of the cell culture used or of the aquatic orqanism.

16. A test kit for performing the method of claim 1, the kit comprising:
a component including recombinant living cells having at least one gene fusion construct, aᵢ-mᵢ, of a gene sequence section, aᵢ, which codes for at least one functional protein, Aᵢ, from the family of the ABC transporter proteins, and a marker protein gene, mᵢ, which codes for at least one marker protein, Mᵢ, from the family of the green fluorescent proteins (GFP) which does not influence the coding of the functional protein, Aᵢ, the gene fusion construct, coding for a fusion protein, Aᵢ-Mᵢ, of the functional protein, Aᵢ, and the marker process, Mᵢ.

17. The kit of claim 16, wherein the living cells are derived recombinantly from a non-recombinant aquatic mother organism.

18. The kit of claim 16 wherein the living cells are derived recombinantly from non-recombinant cell cultures or immortalized cell lines.

19. The method of claim 10, wherein the organism is a crustacean, a cnidarian, a sea anemone, a spiny creature, a sponge, a roundworm, or a flatworm.

20. The method of claim 10, wherein the organism is a flatworm Macrostonum lignano.
